(19) United States
(12) Patent Application Publication
(10) Pub. No.: US 2024/0350071 A1
(43) Pub. Date: Oct. 24, 2024

(54) COMPUTER-IMPLEMENTED METHOD AND SYSTEM FOR GENERATING A NEUROFEEDBACK SIGNAL FOR A NEUROFEEDBACK SESSION ON LOW POWERED DEVICES

(71) Applicant: Pankhtech India Pvt Ltd, New Delhi (IN)

(72) Inventor: Bhavya Madan, Noida (IN)

(21) Appl. No.: 18/304,428

(22) Filed: Apr. 21, 2023

(51) Int. Cl. A61B 5/375; A61B 5/00

(52) U.S. Cl. CPC A61B 5/375; A61B 5/7275

(57) ABSTRACT

A method for generating a neurofeedback signal for neurofeedback session on low powered devices is disclosed. The method includes (a) capturing one or more bio-signals from one or more electronic devices, comprising one or more digital biomarkers; (b) measuring a baseline activity associated with the captured one or more digital biomarkers during a resting state of the brain; (c) measuring an activity rate based on the one or more digital biomarkers; (d) computing a threshold value at a plurality of time series in the neurofeedback session based on at least one of: the measured activity rate and the one or more digital biomarkers captured during the baseline activity of the brain using an activity model; and (e) outputting of the neurofeedback signal corresponding to the neurofeedback session to the user based on the computed threshold value.

### FIELD OF INVENTION

Embodiments of the present disclosure relate to neurofeedback systems and more particularly relate to a computer-implemented method and system for adjusting a threshold value for generating a neurofeedback signal corresponding to a neurofeedback session on low powered devices.

### BACKGROUND

Neurofeedback is a method of informing a user of their brain activity in real time via a sensory feedback. To achieve the neurofeedback, an electroencephalogram (EEG) of the brain is captured in desired frequency ranges from appropriate brain locations and processed to generate a feedback signal. The generated feedback signal can be at least one of: an auditory, a visual or a haptic, and the like in nature. For example, an appearance of a visual cue in a neurofeedback session can be used to indicate rise in an attention level. Thus, the user is able to decipher information that the feedback signal carries. The neurofeedback works similar to operant conditioning, wherein behaviours that are enhanced over multiple training sessions.

Various features of brain function and activity can be trained using the neurofeedback. Some examples are attention, relaxation, resilience to stress and mood. The EEG of the brain is constituted by electromagnetic waves across various frequency ranges. Out of the electromagnetic waves, only those that are relevant to a feature of interest are selected as a biomarker. Further, the biomarker is averaged over a short initial time duration to derive average power of a waveform. During this initial time duration, there is no feedback provided. The average power is used to compute a threshold such that the user is having high activity rate whenever the threshold is crossed. The threshold is further altered to increase or decrease a difficulty level during the neurofeedback training session. The threshold could be regulated manually or using automation. Adjustment of the threshold becomes pivotal to make the session engaging and effective, if too hard, then the neurofeedback training session can discourage the user and if too easy, then the neurofeedback training session might lead to boredom.

The manual thresholding is performed by a neurofeedback specialist who monitors the biomarker levels and changes the threshold whenever the user crosses the threshold by a certain difference for a certain time duration. The manual thresholding can be inconsistent if not done under the same neurofeedback specialist and is further limited by the lack of trained personnel.

The automated thresholding is applied by averaging the biomarker over an overlapping or nonoverlapping time-window. The threshold is computed for each window and is converted into an activity rate administered at a certain rate (typically set at 70-80%). This is the biggest drawback of conventional methods, i.e., the feedback is based on a rate instead of the actual values of a digital biomarker. Usually, the real-time value of the digital biomarkers is in the range of normative distribution and a professional practitioner would not change the threshold letting the user work for the feedback. Further, fixing the activity rate does not allow conventional methods to take signal quality into account making those susceptible to noise artefacts. These caveats lead to the following issues: some of the users are not able to successfully change or train their brain activity, and the change in the brain activity happening in a direction opposite to an intended one, thus defeating the purpose of the neurofeedback.

A research group (Dhindsa, K., Gauder, K. D., Marszalek, K. A., Terpou, B., & Becker, S. (2018) recently attempted to use variable activity rates by employing asymmetric thresholding designed to be biased towards increasing difficulty with a title of “Progressive thresholding: shaping and specificity in automated neurofeedback training” that was published in Institute of Electrical and Electronics Engineers (IEEE) Transactions on Neural Systems and Rehabilitation Engineering, 26 (12), 2297-2305). However, the above said technique passively provides a feedback based on statistical modelling and does not actively optimise the activity rate from real-time biomarker values. Thus, there is a need and scope to innovate further to solve the issues of fixed activity rates.

Therefore, in order to address the aforementioned issues, there is a need for an improved computer-implemented method and system for adjusting a threshold value for generating a neurofeedback signal corresponding to a neurofeedback session on low powered devices.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computer-implemented system for generating a neurofeedback signal for a neurofeedback session is disclosed. The system includes one or more hardware processors and a memory coupled to the one or more hardware processors. The memory includes a plurality of subsystems. The plurality of subsystems includes a data capturing subsystem configured to capture one or more bio-signals from one or more electronic devices, comprising one or more digital biomarkers. The one or more digital biomarkers indicates brain activities of a user. The one or more brain activities of the user includes rhythms and function of a brain.

The plurality of subsystems further includes an activity rate measurement subsystem configured to measure a baseline activity associated with the captured one or more digital biomarkers during a resting state of the brain. The activity rate measurement subsystem is further configured to measure an activity rate based on one or more digital biomarkers. The activity rate includes a positive feedback provided to the user when an activity level of the brain is increased. The plurality of subsystems further includes a threshold computing subsystem configured to compute a threshold value at a plurality of time series in the neurofeedback session based on at least one of: the measured activity rate and one or more digital biomarkers captured during the baseline activity of the brain using an activity model. The plurality of subsystems further includes a neurofeedback output subsystem configured to output of the neurofeedback signal corresponding to the neurofeedback session to the user, based on the computed threshold value.

In an embodiment, in computing the threshold value, the threshold computing system, using the activity model, is configured to compute mean and standard deviation for each window. The window in which one or more digital biomarkers are stored. The threshold computing system is further configured to detect a presence of an artifact in a biomarker list for the window. The mean value and an adjacent factor are applied when the artifact is detected in the biomarker list. Applying of the mean value and the adjacent factor is ignored when recalibration for the one or more digital biomarkers is performed. The adjacent factor is related to a list of weights computed for weighted average of the biomarker list. The threshold computing subsystem is further configured to add a real-time value of a current biomarker to the biomarker list. A list of differences between biomarker values in the biomarker list and current threshold value is determined using the adjacent factor.

The threshold computing subsystem is further configured to determine whether the current biomarker continuously exceeds a second threshold value of a statistical range for more than recalibration count. The recalibration count is related to a timer before recalibration is performed. The threshold computing subsystem is further configured to perform the recalibration for one or more digital biomarkers when the current biomarker continuously exceeds the second threshold value of the statistical range for more than the recalibration count. The threshold computing subsystem is further configured to compute mean and standard deviation for a next window when the current biomarker is within the second threshold value of the statistical range.

In another embodiment, the recalibration for one or more digital biomarker includes assigning weightage to the biomarker values based on order in the plurality of time series, computing a weighted average for the biomarker values to identify the window to be prioritized for computing a new threshold value, and applying the computing new threshold value for the window including one or more digital biomarkers for measuring the activity rate. The most recent digital biomarker value is assigned with higher weightage. The window includes a weighted average that is farthest from an initial threshold value obtains the highest priority.

In yet another embodiment, the initial threshold value is computed from a list of the one or more digital biomarkers in the baseline activity. In yet another embodiment, the initial threshold value at an initial time is a difference of mean values of the one or more digital biomarkers and X times standard deviation of the values of the one or more digital biomarkers. The X times refer to Z-value from normal distribution that is computed based on the measured activity rate. In yet another embodiment, the Z-value from normal distribution is computed from a standard table for the measured activity rate. The standard table is at least one of: a look-up table, and a hash table.

In one aspect, a computer-implemented method for generating a neurofeedback signal for a neurofeedback session is disclosed. The method includes capturing one or more bio-signals from one or more electronic devices, comprising one or more digital biomarkers. The one or more digital biomarkers indicates one or more brain activities of a user. The one or more brain activities of the user includes rhythms and function of a brain. The method further includes measuring a baseline activity associated with the captured one or more digital biomarkers during a resting state of the brain.

The method further includes measuring an activity rate based on one or more digital biomarkers. The activity rate includes a positive feedback provided to the user when an activity level of the brain is increased. The method further includes computing a threshold value at a plurality of time series in the neurofeedback session based on at least one of: the measured activity rate and the one or more digital biomarkers captured during the baseline activity of the brain using an activity model. The method furthermore includes outputting the neurofeedback signal corresponding to the neurofeedback session to the user based on the computed threshold value.

In another aspect, a non-transitory computer-readable storage medium having instructions for generating a neurofeedback signal for a neurofeedback session is disclosed. The non-transitory computer-readable storage medium having the instructions stored therein that when executed by one or more hardware processors, cause one or more hardware processors to execute operations of: capturing one or more bio-signals representing as one or more digital biomarkers: measuring a baseline activity associated with the captured one or more digital biomarkers during a resting state of the brain: measuring an activity rate based on the one or more digital biomarkers, wherein the activity rate comprises a positive feedback provided to the user when an activity level of the brain is increased: computing a threshold value at a plurality of time series in the neurofeedback session based on at least one of: the measured activity rate and the one or more digital biomarkers captured during the baseline activity of the brain using an activity model; and outputting the neurofeedback signal corresponding to the neurofeedback session to the user based on the computed threshold value.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computer-implemented system for dynamically adjusting a threshold value for generating a neurofeedback signal corresponding to a neurofeedback session, in accordance with an embodiment of the present disclosure;

FIG. 2 is a detailed view of the computer-implemented system, in accordance with an embodiment of the present disclosure:

FIG. 3 is an exemplary representation illustrating a startup screen of a mobile application module, in accordance with an embodiment of the present disclosure:

FIGS. 4A-4B is an exemplary representation illustrating a selecting mood for the user in the mobile application module, in accordance with an embodiment of the present disclosure:

FIGS. 5A-5C is an exemplary representation illustrating a device discovery and connection screen to connect with a headband, in accordance with an embodiment of the present disclosure:

FIGS. 6A-6B is an exemplary representation illustrating the mobile application module showing the neurofeedback session, in accordance with an embodiment of the present disclosure:

FIGS. 7A-7D is an exemplary representation illustrating the mobile application module showing stability of sensor connectivity, in accordance with an embodiment of the present disclosure:

FIGS. 8A-8B is an exemplary representation illustrating the mobile application module during computation of PDR of the user's brain, in accordance with an embodiment of the present disclosure:

FIGS. 9A-9B is an exemplary representation illustrating the mobile application module during the neurofeedback session, in accordance with an embodiment of the present disclosure:

FIG. 10 is an exemplary representation illustrating the mobile application module showing insights from the neurofeedback session, in accordance with an embodiment of the present disclosure:

FIG. 11 is a graphical representation illustrating an exemplary biomarker array over time period with threshold changing with the present system, in accordance with an embodiment of the present disclosure; and

FIG. 12 is a flow chart illustrating a computer-implemented method for dynamically adjusting a threshold value for generating a neurofeedback signal corresponding to neurofeedback session on low powered devices, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

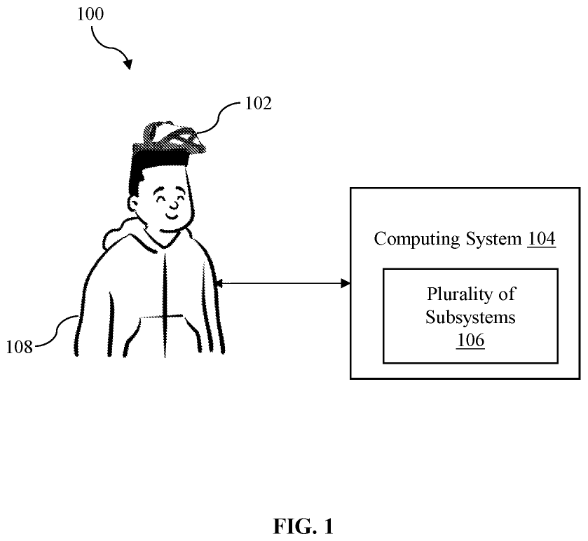

Referring now to the drawings, and more particularly to FIG. 1 through FIG. 12, where similar reference characters denote corresponding features consistently throughout the figures, there are shown preferred embodiments and these embodiments are described in the context of the following exemplary system and/or method.

### Definitions

Electroencephalogram (EEG): An Electroencephalogram (EEG) refers to an electrical activity that is recorded from a brain. The EEG generates from the electrical signals that travel through brain cells and a sum of the electrical signals gives rise to brainwaves. The brain waves span the frequency ranges from as low as 1 Hz up to 100 Hz. The range is majorly divided into following frequency bands: delta (1-4 Hz), theta (4-8 Hz), alpha (8-12 Hz), beta (12-35 Hz), gamma (35-100 Hz). There exist more frequency bands that overlap with these majorly known categories.

Brain activity: a brain activity refers to rhythms and functioning of the brain throughout a sleep-wake cycle. An absence of the brain activity denotes a dead brain. One of the methods to record and study the brain activity is using the EEG.

Baseline state: a baseline state or a baseline phase refers to a duration during which resting activity of the brain persists.

Resting activity: a resting activity refers to the brain activity when a person is not engaged in any defined task and is idly sitting or lying down with eyes open or closed. During the resting state, the person is not even engaged in any mental task such as deliberately focusing on a thought. The resting state or resting phase refers to a duration during which the resting activity of the brain persists.

Baseline activity: a baseline activity refers to the recorded EEG during the resting state of the brain.

Biomarker: a biomarker or a bio-signal refers to any parameter that provides information on a physiological aspect of a body. For example, blood glucose levels are a biomarker that inform the person about absence, presence or risk of diabetes. In the present disclosure, the biomarker is used interchangeably with neuro marker as all the biomarkers in the present disclosure are derived from the EEG of the brain, which is a part of the body's nervous system.

Digital biomarker: a digital biomarker refers to a digital fingerprint of a real biomarker that is recorded without requiring isolation of biological samples like body fluids. The digital biomarkers are recorded through appropriate sensors directly from the body. Usually, these digital biomarkers are constituted of signals that the body naturally emits such as electrical, thermal or mechanical signals. For example, the EEG of the brain is a biomarker, and the properties of EEG derived after some amount of computational processing are digital biomarkers, such as band power in certain frequency ranges or ratios of some of these band powers.

Sensor: a sensor refers to dry EEG electrodes that when in direct contact with the scalp, conduct and send an electrical activity from the brain to an amplifier and a recording unit.

Threshold: a threshold refers to a minimum value that the biomarker must attain in order to trigger the implementation of an algorithm used in the present disclosure.

Neurofeedback: a neurofeedback refers to a process of recording neuro markers such as an EEG signal, real-time to understand how the brain responds to certain stimuli, and then learning to alter the brain activity through a constant feedback presented in a form a sensory signal. For example, when a user is advised to work on training the beta power to increase to achieve higher attention levels. Every time the user's EEG shows a rise in beta band power, a visual cue is presented. The user is told that the visual cue means a rise in attention level. Now the user is supposed to maintain thoughts or activities that help in maximising the presentations of the visual cue. In the example, the visual cue is an activity rate (i.e., based on an activity of the brain) on achieving high attention: an absence or a presence of the activity rate is the real-time feedback on attention level; and the beta band power is the digital biomarker.

Real-time: a real-time refers to a continuously repeating process, wherein a shortest gap between two iterations is small enough to be unnoticeable and yet most effective in achieving an intended goal.

Artifact: an artefact is any noise that mimics a signal or is erroneously interpreted as an actual signal.

Headband: a headband refers to a dry EEG recording device. This headband is crucial in the neurofeedback process as the headband records the EEG to help a mobile application that generates a real-time feedback.

Operant conditioning: an operant conditioning refers to changing a behaviour based on a memory and learning from its past consequences.

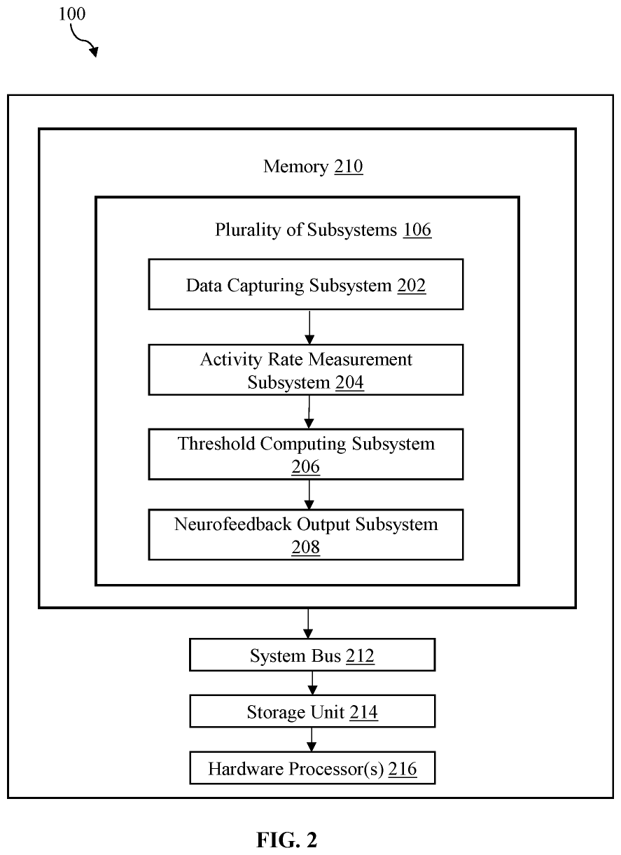

FIG. 1 is a block diagram illustrating an exemplary computer-implemented system 100 for dynamically adjusting a threshold value for generating a neurofeedback signal corresponding to a neurofeedback session, in accordance with an embodiment of the present disclosure. According to FIG. 1, the computer-implemented system 100 includes a headband 102, a user 108 and a computing system 104. The headband 102 is connected to user's 108 head. In an embodiment of the present disclosure, the computing system 104 may be a server or a cloud. The computing system 104 computes a threshold value and transmits the computed threshold value to an electronic device of the user 108. In an exemplary embodiment of the present disclosure, the computing system 104 may include one or more electronic devices. The one or more electronic devices may include a laptop computer, a desktop computer, a tablet computer, a Smartphone, and the like. In an embodiment of the present disclosure, the computing system 104 includes a local browser, a mobile application or a combination thereof. Furthermore, the user 108 may use a web application via the local browser, the mobile application or a combination thereof to communicate with the headband 102. In an embodiment of the present disclosure, the computing system 104 includes a plurality of subsystems 106. Details on the plurality of subsystems 106 have been elaborated in subsequent paragraphs of the present description with reference to FIG. 2.

FIG. 2 is a detailed view of the computer-implemented system 100, in accordance with an embodiment of the present disclosure. According to FIG. 2, the computer-implemented system 100 includes the plurality of subsystems 106, a memory 210, a system bus 212, a storage unit 214, and one or more hardware processors 216.

The memory 210 comprises the plurality of subsystems 106 in the form of programmable instructions executable by the one or more hardware processors 216. Further, the plurality of subsystems 106 includes a data capturing subsystem 202, an activity rate measurement subsystem 204, a threshold computing subsystem 206 and a neurofeedback output subsystem 208.

The one or more hardware processors 216, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 216 may also include embedded controllers, such as generic or programmable logic devices or arrays, application specific integrated circuits, single-chip computers, and the like.

The memory 210 may be non-transitory volatile memory and non-volatile memory. The memory 210 may be coupled for communication with the one or more hardware processors 216, such as being a computer-readable storage medium. The one or more hardware processors 216 may execute machine-readable instructions and/or source code stored in the memory 210. A variety of machine-readable instructions may be stored in and accessed from the memory 210. The memory 210 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 210 includes the plurality of subsystems 106 stored in the form of machine-readable instructions on any of the above-mentioned storage media and may be in communication with and executed by the one or more hardware processors 216.

The storage unit 214 may be a cloud storage, or a location on a file system directly accessible by the plurality of subsystems 106. The storage unit 214 may store one or more bio-signals.

The plurality of subsystems 106 include the data capturing subsystem 202 that is communicatively connected to the one or more hardware processors 216. The data capturing subsystem 202 is configured to capture the one or more bio-signals from the one or more electronic devices, representing as one or more digital biomarkers. The one or more digital biomarkers indicates one or more brain activities of the user 108. The one or more brain activities of the user 108 comprises rhythms and function of a brain. The digital biomarkers refer to a digital fingerprint of a real biomarker that is recorded without requiring isolation of biological samples like body fluids. The digital biomarkers are recorded through appropriate sensors directly from the body. Usually, these digital markers comprise the signals that the body naturally emits such as electrical, thermal or mechanical signals. For example, the EEG of the brain is a biomarker, and the properties of the EEG derived after some amount of computational processing are digital biomarkers, such as band power in certain frequency ranges or ratios of some of these band powers.

The plurality of subsystems 106 include the activity rate measurement subsystem 204 that is communicatively connected to the one or more hardware processors 216. The activity rate measurement subsystem 204 is configured to measure a baseline activity associated with the captured one or more digital biomarkers during a resting state of the brain. Further, the activity rate measurement subsystem 204 is configured to measure an activity rate based on the one or more digital biomarkers. The activity rate includes to a positive feedback provided to the user 108 when an activity level of the brain is increased. The baseline activity is related to a recorded EEG during the resting state of the brain.

In an embodiment of the present disclosure, during the baseline phase of the neurofeedback session, an initial threshold is computed. For example, if the activity rate is 90%, Z-value from normal distribution will be 1.28. The Z-value from normal distribution is computed from a standard table for the measured activity rate. The standard table is at least one of: a look-up table, and a hash table. In an embodiment of the present invention, the digital biomarker is a band power. The list of digital biomarkers in the baseline phase threshold at time zero is as follows:

thr(0)=mean(band power)−1.28*std dev(band power) equation (1)

The plurality of subsystems 106 includes the threshold computing subsystem 206 that is communicatively connected to the one or more hardware processors 216. The threshold computing subsystem 206 is configured to automatically compute a threshold value at a plurality of time series in the neurofeedback session based on at least one of: the measured activity rate and one or more digital biomarkers captured during the baseline activity of the brain using an activity model. In an embodiment, the activity model may include at least one of: an artificial intelligence (AI) model and a machine learning (ML) model. In an embodiment, the threshold computing subsystem 206 automatically computes the threshold value at the plurality of time series in the neurofeedback session based on at least one of: the measured activity rate and one or more digital biomarkers captured during the baseline activity of the brain, using at least one of: a Linear Regression model, a deep neural networks (CNN) model, a convolutional neural network (CNN) model, a decision trees model, a random forest model, and the like. The threshold value is related to a minimum value that the biomarker must attain in order to trigger the implementation of the model used in the present disclosure.

In an embodiment of the present disclosure, the threshold computing subsystem 206, using the activity model, computes a mean and standard deviation for each window. The window in which one or more digital biomarkers are stored. The threshold computing subsystem 206 further detects a presence of an artefact in a biomarker list for the window. The mean value and an adjacent factor are applied when the artifact is detected in the biomarker list. Applying of the mean value and the adjacent factor is ignored when recalibration for the one or more digital biomarkers is performed. In an embodiment, the adjacent factor is related to a list of weights computed for weighted average of the biomarker list. The threshold computing subsystem 206 further adds a real-time value of a current biomarker to the biomarker list. The list of differences between biomarker values in the biomarker list and a current threshold value is determined using the adjacent factor.

The threshold computing subsystem 206 further determines whether the current biomarker is continuously outside the statistical range (i.e., the current biomarker exceeds a second threshold value of the statistical range) for more than recalibration count. The recalibration count is related to a timer before recalibration is performed. The threshold computing subsystem 206 further performs recalibration for the one or more digital biomarkers when the current biomarker is continuously outside the statistical range for more than the recalibration count. The threshold computing subsystem 206 further computes mean and standard deviation for a next window when the current biomarker is within the statistical range for more than the recalibration count.

In an embodiment of the present disclosure, the recalibration for the one or more digital biomarkers comprises assigning weightage to the biomarker values based on an order in the plurality of time series. In an embodiment, the most recent digital biomarker value is assigned with higher weightage. The recalibration for the one or more digital biomarkers further comprises computing a weightage average for the biomarker values to identify the window to be prioritized for computing a new threshold value. The window whose weighted average is farthest from an initial threshold value obtains the highest priority. The recalibration for one or more digital biomarkers further comprises applying the computed new threshold value for the window including the one or more digital biomarkers for measuring the activity rate.

In an embodiment of the present disclosure, the variable used in the present activity model is provided. For example, ‘recal_cnt’ is timer before recalibration is called, default 10. Further, ‘win_size’ is window size for which biomarkers are stored, too big window size causes a delay and too small window size decreases the efficiency of the system. Further, ‘biomarker’ is a list of biomarkers for last window size. Furthermore, ‘adj_factor’ is list of weights for weighted average of biomarker list. Below is the pseudo code (1)—

| if(artefact) | |
|---|---|
| biomarker.push(band_mean) | |
| adj_factor.push(0) | |
| else | |
| biomarker.push(band_power(t)) | |
| biomarker.pop(0) | |
| adj = mod(biomarker[t] − Thr[t−1]) | |
| adj_factor.push(adj[t]) | |
| adj_factor.pop(0) | ....computer instruction (1) |

In an embodiment of the present disclosure, the threshold computing subsystem 206, using the activity model, compares if the current biomarker is continuously outside the statistical range for more than recal_cnt, then the recalibrate function will be called, else the method will go back to step 1. Below is the pseudo code (2)—

```
if(biomarker[t] > 4 * std_dev(band power))
  recal_count = recal_count − 1
if(!recal_count)
  recalibrate (biomarker, adj_factor) ....computer instruction (2)
```

In an embodiment of the present disclosure, the weighted average of the biomarkers is computed to identify the windows which has to be prioritized for new threshold computation, using the activity model. Below is the pseudo code (3)—

| recalibrate(biomarker, adj_factor) | |
|---|---|
| dis_rate = 1 − mod[(Thr(t−1) − biomarker[t])/Thr(t−1)] | |
| for i in range (9,0) | |
| dis_factor[i] = 1/(1 + dis_rate)\hat{ }(9−i) | |
| w_sum =0 | |
| for in range (0,9) | |
| if(dis_factor[i] > 0.1) | |
| w[i] = dis_factor[i]*adj_factor[i] | |
| w_sum = w_sum + w[i] | |
| else | |
| w[i] = 0 | .... computer instruction (3) |

Thr[t]=dotproduct(biomarker,w[i])/w_sum

In an embodiment of the present disclosure, example for threshold below power is as follows in below pseudo code (4)—

let Thr[0] = 10, std dev = 0.2
biomarker = [9,8,7,6,6,6,6,7,8,8]
adj_factor = [1,2,3,4,4,4,4,3,2,2]
“biomarker[t] > 4 * std_dev(band power)” is true for all so
recalibration is called
dis_rate = 1 − (10−8)/10 = 0.8
dis_factor =
[0.002800753897,0.005041357015,0.009074442627,0.01633399673,
0.02940119411,0.0529221494,0.1714677641,0.3086419753,0.555555
5556,1]
w = [0,0,0,0,0,0,.684,.93,1.1,2]
w_sum = 4.714

| | |
|---|---|
| Thr[t] = 7.51 | .... computer instruction (4) |

In an embodiment of the present disclosure, an example for the threshold above power is as follows in below pseudo code (5)—

| let Thr[0] = 10, std dev = 0.2 | |
|---|---|
| biomarker = [11,11,12,12,12,13,14,15,16,15] | |
| adj_factor = [1,1,2,2,2,3,4,5,6,5] | |
| “biomarker[t] > 4 * std_dev(band power)” is true for all so | |
| recalibration is called | |
| dis_rate = 1 − mod(10−15)/10 =0.5 | |
| dis_factor = [0.03,0.04,0.06,0.09,0.13,0.20,0.30,0.44,0.66,1] | |
| w = [0,0,0,0,0.26,0.6,1.2,2.2,3.96,5] | |
| w_sum = 13.22 | |
| Thr[t] = 15.75 | .... computer instruction (5) |

The plurality of subsystems 106 includes the neurofeedback output subsystem 208 that is communicatively connected to the one or more hardware processors 216. The neurofeedback output subsystem 208 is configured to output the neurofeedback signal corresponding to the neurofeedback session to the user 108 based on the computed threshold value. The output signal will be displayed through a display of the computing system 104.

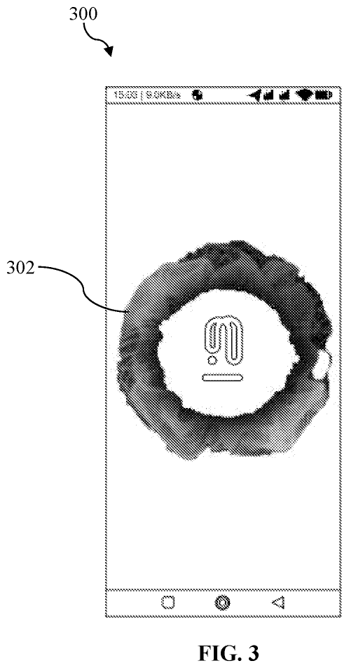

FIG. 3 is an exemplary representation 300 illustrating a startup screen 302 of a mobile application module, in accordance with an embodiment of the present disclosure. According to FIG. 3, the exemplary representation 300 shows the mobile application module. The user 108 needs to start the mobile application module for further processes of the neurofeedback session.

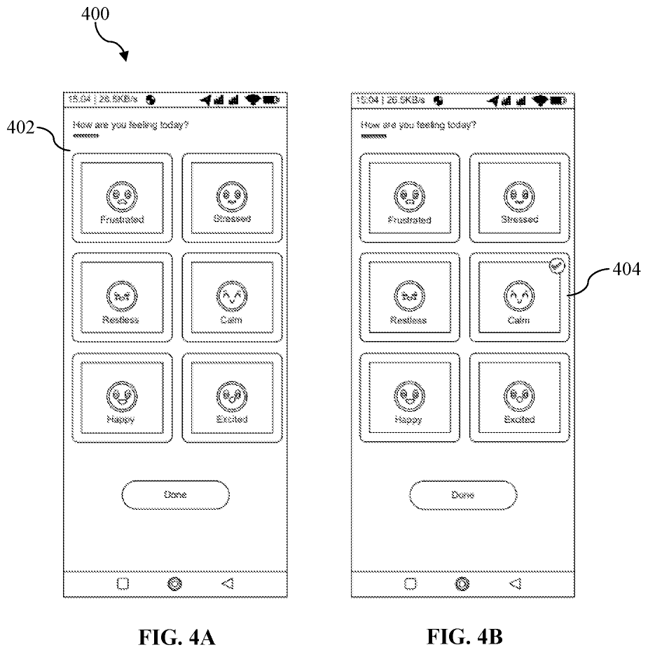

FIGS. 4A-4B is an exemplary representation 400 illustrating a selecting mood 404 for the user 108 in the mobile application module, in accordance with an embodiment of the present disclosure. Referring to FIGS. 4A-4B, the user 108 gets an option 402 to select the mood 404. The options 402 enable the user 108 to select the user mood 404 that include at least one of: frustrated, stressed, restless, calm, happy, excited, and the like, mood 404 of the user 108.

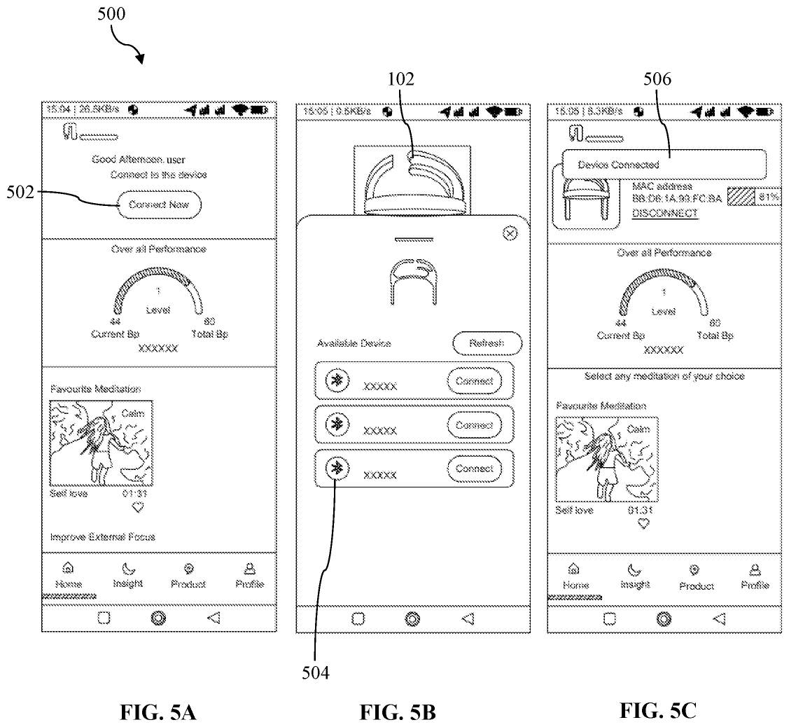

FIGS. 5A-5C are exemplary representations 500 illustrating an exemplary device discovery and connection screen to connect with the headband 102, in accordance with an embodiment of the present disclosure. According to FIGS. 5A-5C, in the mobile application module, the screen is displayed when the user 108 clicks on ‘connect now’ option 502 to connect the mobile application module with the headband 102. The headband 102 comprises an input and an output button on an outer left surface. A Light Emitting Diode (LEDs) light up indicates that the headband 102 is in ON state. The headband 102 and the mobile application module are connected via a Bluetooth 504. Once the Bluetooth is on, all the headbands which are switched on are discovered and displayed on the screen of the mobile application module. Once the user 108 clicks on the ‘connect’ option, the mobile application module will connect to the headband 102. The mobile application module displays a notification 506 over the successful connection.

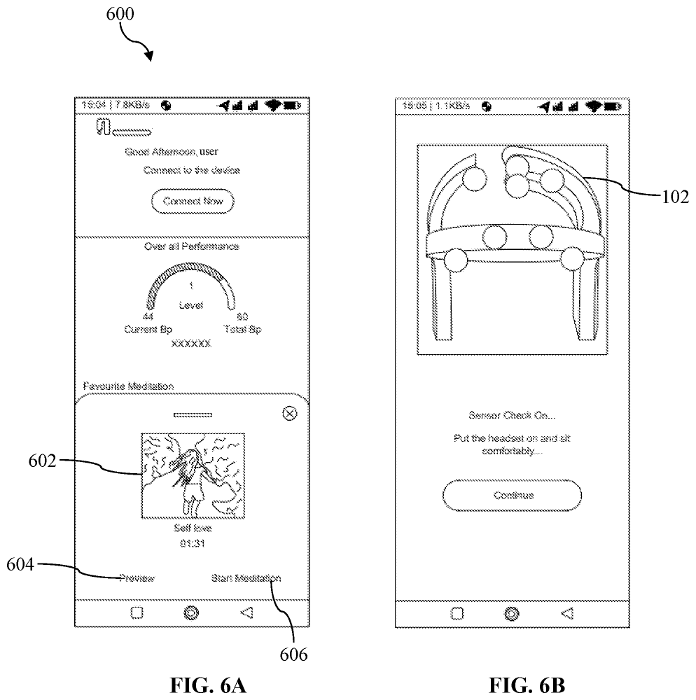

FIGS. 6A-6B are exemplary representations 600 illustrating the mobile application module showing the neurofeedback session, in accordance with an embodiment of the present disclosure. According to FIG. 6A, the user 108 selects a meditation music 602 based on his/her mood 404 from a list provided in the mobile application module. For example, the user 108 selects a ‘self love’ meditation 602 on the mobile application module. When the ‘self love’ option 602 is clicked by the user 108 then the user 108 will get an option for preview 604 and start meditation 606 on the screen. According to FIG. 6B, the mobile application module checks whether the user wears the headband 102, and instructs the user 102 to wear the headband 102 and to turn the headband 102 ON.

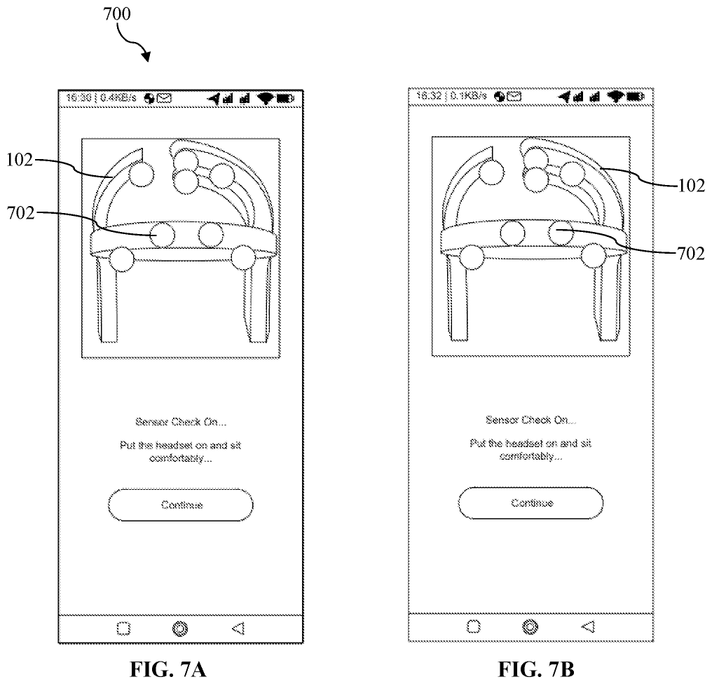

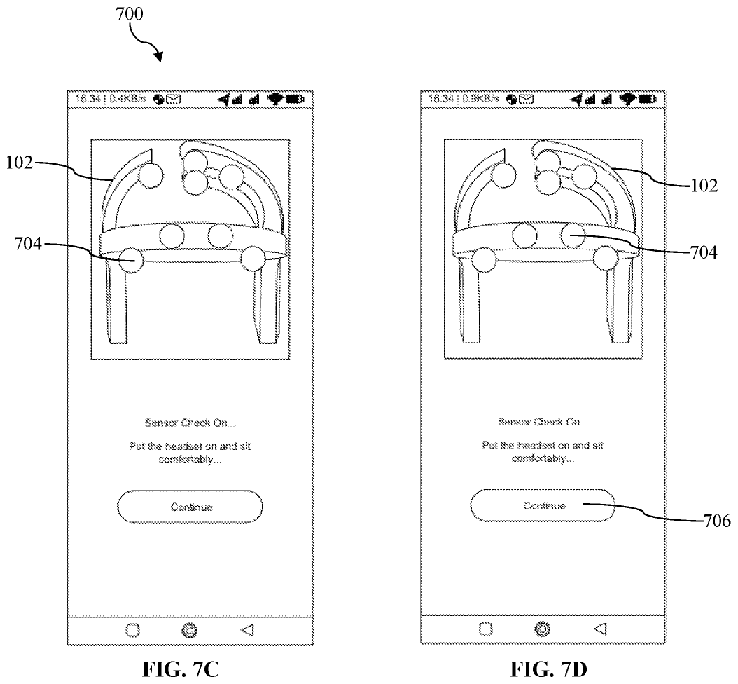

FIGS. 7A-7D are exemplary representations 700 illustrating the mobile application module showing a stability of sensor connectivity, in accordance with an embodiment of the present disclosure. According to FIGS. 7A-7D, circles 702 (e.g., red circles) are overlapped upon the headband 102 represents locations of all sensors in the headband 102. A red colour 702 indicates that a sensor is not yet stably connected to the scalp of the user 108. Circles 704 turn green when a stable sensor to scalp connection is established. The user 108 then uses a map of the established connection as a reference to adjust the headband 102 ensuring each sensor directly contacts the skin of the scalps. Sometimes it is required to brush aside the hair to expose the scalp at the required regions for better connection establishment between head of the user 108 and the headband 102. Once all sensors turn green, the user 108 clicks on ‘continue’ option 706 to further processes. Further, the user 108 will have an option of moving forward with the meditation if at least Fz and Pz sensors are connected. The Fz and Pz sensors are connected in the centre on the top of the headband 102. In an embodiment, the Fz and Pz sensors (i.e., electrodes) are placed on a midline sagittal plane of the skull for reference/measurement points. The Fz sensor captures the brain activity from a frontal cortical region and Pz sensor captures the brain activity from a parietal cortical region, both on the midline plane.

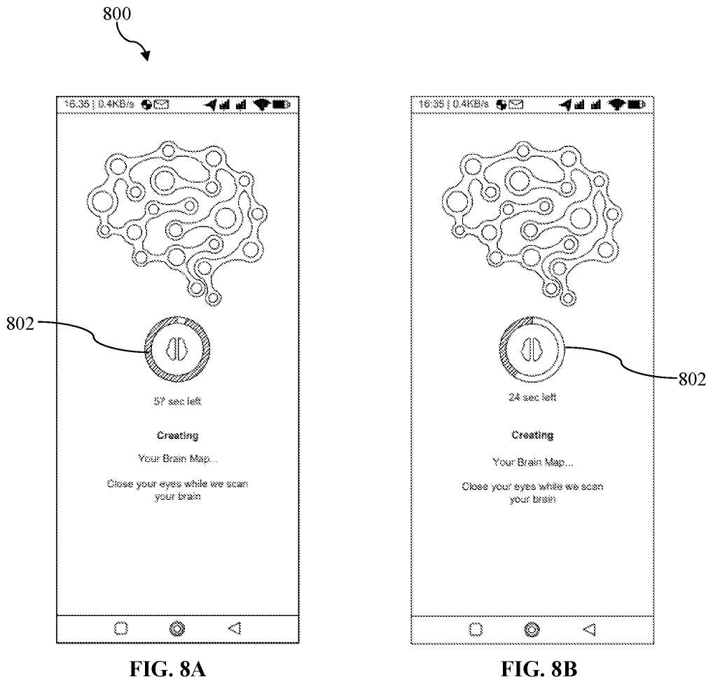

FIGS. 8A-8B are exemplary representations 800 illustrating the mobile application module during a computation of PDR of the user's brain, in accordance with an embodiment of the present disclosure. According to FIGS. 8A-8B, on clicking a continue button on the mobile application module, the screen appears and the user 108 needs to sit with closed eyes during the neurofeedback session. The 60 second running timer 802 shows the computation of a posterior dominant rhythm (PDR) progress. Following the 60 seconds, the selected meditation music will start to play.

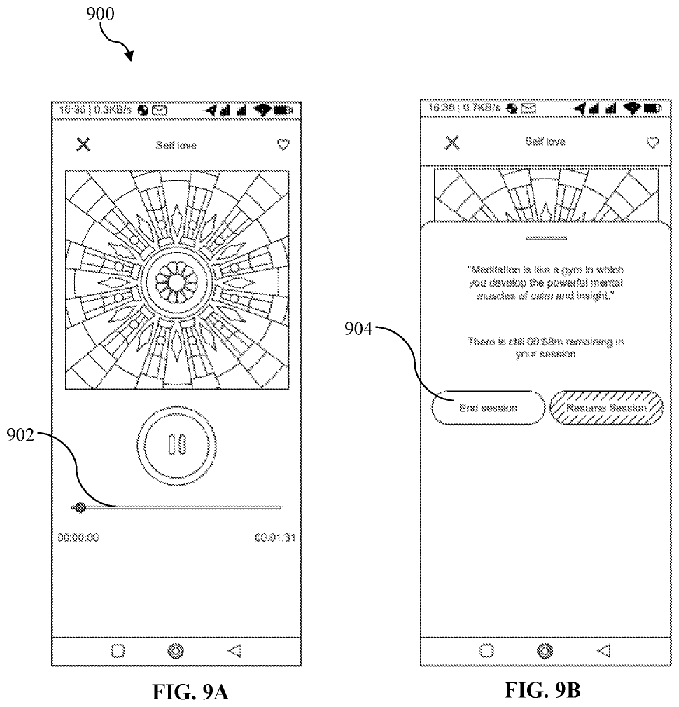

FIGS. 9A-9B are exemplary representations 900 illustrating the mobile application module during the neurofeedback session, in accordance with an embodiment of the present disclosure. According to FIGS. 9A-9B, in the screen, a music duration 902 is 1 minute and 31 seconds. During this neurofeedback session, the biomarker is captured and presented as an input signal to the application running in background. The automated thresholding is applied on this input and then the output is produced based on the applied input. The output is provided to the user 108 in the form of volume modulation of the playing music. During each neurofeedback session, the user 108 experiences the neurofeedback signal which is adjusted in magnitude as per the input signal. The neurofeedback output is provided over the entire duration 902 of the music. The user 108 chooses to end the session 904 earlier if needed and thereby stop the neurofeedback experience.

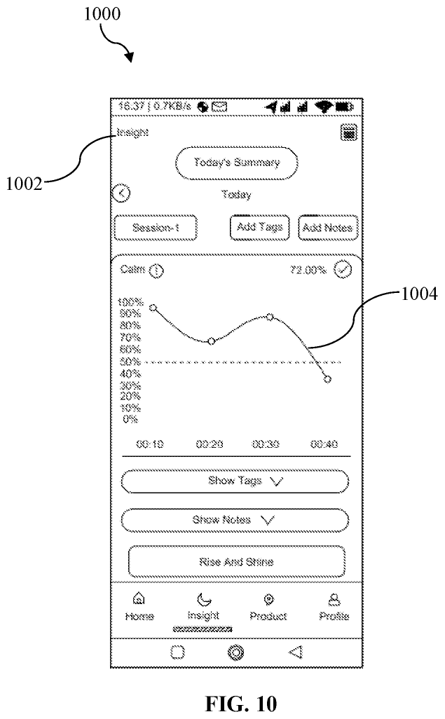

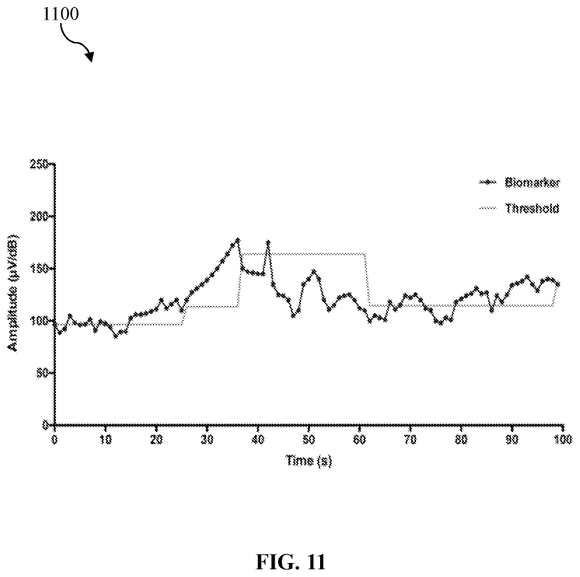

FIG. 10 is an exemplary representation 1000 illustrating the mobile application module showing insights 1002 from the neurofeedback session, in accordance with an embodiment of the present disclosure. According to FIG. 10, once the neurofeedback session ends, the user 108 is able to view a graphical plot 1004 of how the biomarker varied during the entire session and various other insights derived from user's 108 EEG data.

FIG. 11 is a graphical representation 1100 illustrating an exemplary biomarker array over time period with threshold changing with a present system, in accordance with an embodiment of the present disclosure. According to FIG. 11, a representative biomarker array (i.e., marked in line with dots) over 99 seconds long period along with the threshold (i.e., marked in line only) changing via the present system. In the example, the recalibration count is 4 seconds, i.e., the next threshold will be computed at least 4 seconds after applying the previous threshold. The window size is 10 seconds, i.e., the new threshold is computed from 10 seconds long window.

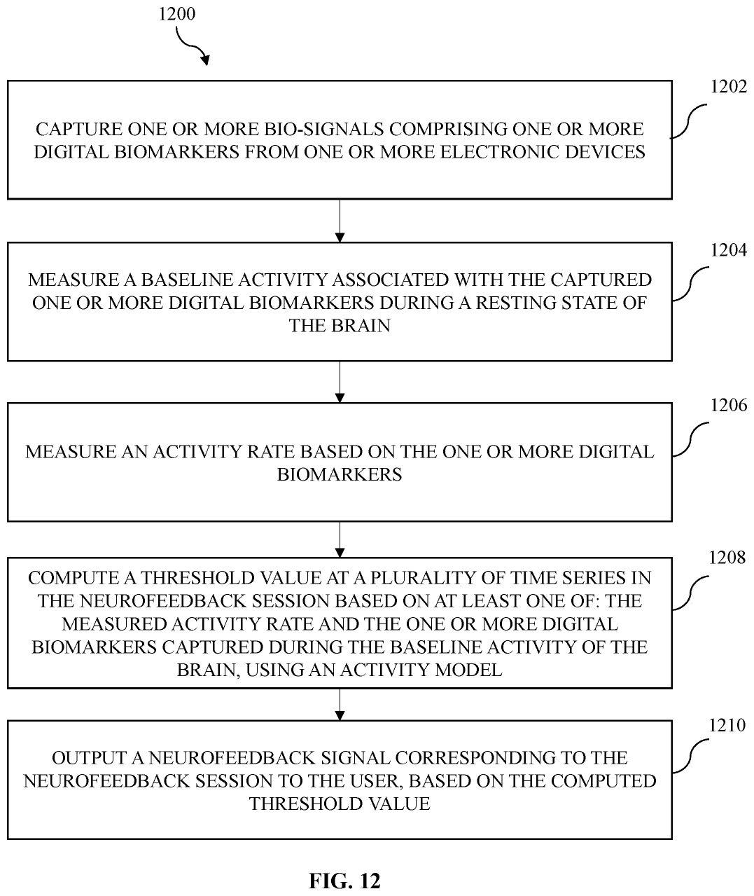

FIG. 12 is a flow chart illustrating a computer-implemented method 1200 for dynamically adjusting the threshold value for generating a neurofeedback signal corresponding to the neurofeedback session on low powered devices, in accordance with an embodiment of the present disclosure. According to FIG. 12, at step 1202, one or more bio-signals are captured from one or more electronic devices, as one or more digital biomarkers. The one or more digital biomarkers indicates the one or more brain activities of the user 108. The one or more brain activities of the user 106 comprises the rhythms and function of the brain.

At step 1204, the baseline activity associated with the captured one or more digital biomarkers during the resting state of the brain is measured.

At step 1206, the activity rate is measured based on one or more digital biomarkers. The activity rate includes the positive feedback provided to the user 108 when the activity level of the brain is increased.

At step 1208, the threshold value is computed at the plurality of time series in the neurofeedback session based on at least one of the measured activity rate and the one or more digital biomarkers captured during the baseline activity of the brain using the activity model.

At step 1210, the neurofeedback signal corresponding to the neurofeedback session to the user 108 is outputted based on the computed threshold value.

In an embodiment of the present disclosure, the automatic computation of the threshold value, using the activity model, includes computing the mean and standard deviation for each window. The window in which the one or more digital biomarkers are stored. The computation of the threshold value, using the activity model, further comprises detecting the artifact in the biomarker list for the window. The mean value and the adjacent factor are applied when the artifact is detected in the biomarker list. Applying the mean value and the adjacent factor is ignored when recalibration for the one or more digital biomarkers is performed and the adjacent factor that is related to the list of weights computed for weighted average of the biomarker list.

The computation of the threshold value, using the activity model, further comprises adding the real-time value of the current biomarker to the biomarker list. The list of differences between biomarker values in the biomarker list and a current threshold value is determined using the adjacent factor. The computation of the threshold value, using the activity model, further comprises determining whether the current biomarker is continuously outside the statistical range (i.e., the current biomarker exceeds a second threshold value of the statistical range) for more than recalibration count. The recalibration count is related to a timer before recalibration is performed. The computation of the threshold value, using the activity model, further comprises performing recalibration for the one or more digital biomarkers when the current biomarker is continuously outside the statistical range for more than the recalibration count. The computation of the threshold value, using the activity model, further comprises computing the mean and standard deviation for a next window when the current biomarker is within the statistical range for more than the recalibration count.

In an embodiment of the present disclosure, the recalibration for the one or more digital biomarkers comprises assigning the weightage to the biomarker values based on order in the plurality of time series. The most recent digital biomarker value is assigned with higher weightage. The recalibration for the one or more digital biomarkers further comprises computing the weighted average for the biomarker values to identify the window to be prioritized for computing the new value. The window whose weighted average is farthest from an initial threshold value obtains the highest priority. The recalibration for the one or more digital biomarkers further comprises applying the computed new threshold values for the window having the one or more digital biomarkers for measuring the activity rate.

In an embodiment of the present disclosure, the initial threshold value is computed from the list of the one or more digital biomarkers in the baseline activity.

In an embodiment of the present disclosure, the initial threshold value at the initial time is the difference of mean values of the one or more digital biomarkers and X times standard deviation of the values of the one or more digital biomarkers. The X times refer to the Z-value from normal distribution that is computed based on the measured activity rate.

In an embodiment of the present disclosure, the Z-value from normal distribution is computed from the standard table for the measured activity rate. The standard table is at least one of: a look-up table, and a hash table.

In an embodiment of the present disclosure, the present disclosure helps in automating the effective neurofeedback therapy. This is done by addressing the issues of the fixed activity-rate thresholding, wherein the threshold is too adaptive and unlike the one usually followed by professionals.

In an embodiment of the present disclosure, the present disclosure is designed to be optimized for low power mobile and IoT devices. The present invention is ideal for unlocking a huge market for neurofeedback practitioners as the computer-implemented system 100 provides/leases the hardware to their users and their electronic device is capable enough to do the neurofeedback training without the data ever leaving their devices.

In an embodiment of the present disclosure, the aim of the neurofeedback session is to train the subject's (i.e., the user 108) brain activity in the desired direction. The present disclosure automates the process, where the professional just needs to set few parameters before the session and not track the client response throughout the session. The present computer-implemented method 1200 reduces the number of computations by avoiding computing the threshold for every second, rather recalibrate function is only called if the real time value is statistically out of range for a particular time to adjust the threshold value. The present disclosure is easy to implement on low power devices including mobile phones, IoT devices, and the like.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising.” “having.” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a.” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. A computer-implemented method for generating a neurofeedback signal for a neurofeedback session, the computer-implemented method comprising:
capturing, by one or more hardware processors, one or more bio-signals from one or more electronic devices, comprising one or more digital biomarkers, wherein the one or more digital biomarkers indicates one or more brain activities of a user, and wherein the one or more brain activities of the user comprises rhythms and function of a brain:
measuring, by the one or more hardware processors, a baseline activity associated with the captured one or more digital biomarkers during a resting state of the brain:
measuring, by the one or more hardware processors, an activity rate based on the one or more digital biomarkers, wherein the activity rate comprises a positive feedback provided to the user when an activity level of the brain is increased:
computing, by the one or more hardware processors, a threshold value at a plurality of time series in the neurofeedback session based on at least one of: the measured activity rate and the one or more digital biomarkers captured during the baseline activity of the brain, using an activity model; and
outputting, by the one or more hardware processors, the neurofeedback signal corresponding to the neurofeedback session to the user, based on the computed threshold value.

2. The computer-implemented method as claimed in claim 1, wherein computing the threshold value using the activity model comprises:
computing, by the one or more hardware processors, mean and standard deviation for each window, wherein the window in which the one or more digital biomarkers are stored:
detecting, by the one or more hardware processors, a presence of an artifact in a biomarker list for the window, wherein the mean value and an adjacent factor are applied when the artifact is detected in the biomarker list, wherein applying of the mean value and the adjacent factor is ignored when recalibration for the one or more digital biomarkers is performed, and wherein the adjacent factor is related to a list of weights computed for weighted average of the biomarker list;
adding, by the one or more hardware processors, a real-time value of a current biomarker to the biomarker list, wherein a list of differences between biomarker values in the biomarker list and a current threshold value, is determined using the adjacent factor:
determining, by the one or more hardware processors, whether the current biomarker continuously exceeds a second threshold value of a statistical range for more than recalibration count, wherein the recalibration count is related to a timer before recalibration is performed:
performing, by the one or more hardware processors, the recalibration for the one or more digital biomarkers when the current biomarker continuously exceeds the second threshold value of the statistical range for more than the recalibration count; and
computing, by the one or more hardware processors, mean and standard deviation for a next window when the current biomarker is within the second threshold value of the statistical range.

3. The computer-implemented method as claimed in claim 2, wherein performing the recalibration for the one or more digital biomarkers comprises:
assigning, by the one or more hardware processors, weightage to the biomarker values based on an order in the plurality of time series, wherein the most recent digital biomarker value is assigned with higher weightage:
computing, by the one or more hardware processors, a weighted average for the biomarker values to identify the window to be prioritized, wherein the window is identified for computing a new threshold value, and wherein the window comprises a weighted average that is farthest from an initial threshold value obtains the highest priority; and
applying, by the one or more hardware processors, the computed new threshold value for the window comprising the one or more digital biomarkers for measuring the activity rate.

4. The computer-implemented method as claimed in claim 3, wherein the initial threshold value is computed from a list of the one or more digital biomarkers in the baseline activity.

5. The computer-implemented method as claimed in claim 4, wherein the initial threshold value at an initial time is a difference of mean values of the one or more digital biomarkers and X times standard deviation of the values of the one or more digital biomarkers, wherein the X times refer to Z-value from normal distribution that is computed based on the measured activity rate.

6. The computer-implemented method as claimed in claim 5, wherein the Z-value from normal distribution is computed from a standard table for the measured activity rate, and wherein the standard table is at least one of: a look-up table, and a hash table.

7. A computer-implemented system for generating a neurofeedback signal for a neurofeedback session, the computer-implemented system comprising:
one or more hardware processors; and
a memory coupled to the one or more hardware processors, wherein the memory comprises a plurality of subsystems in the form of programmable instructions executable by the one or more hardware processors, and wherein the plurality of subsystems comprises:
a data capturing subsystem configured to capture one or more bio-signals from one or more electronic devices, comprising one or more digital biomarkers, wherein the one or more digital biomarkers indicates one or more brain activities of a user, and wherein the one or more brain activities of the user comprises rhythms and function of a brain:
an activity rate measurement subsystem configured to:
measure a baseline activity associated with the captured one or more digital biomarkers during a resting state of the brain;
measure an activity rate based on the one or more digital biomarkers, wherein the activity rate comprises a positive feedback provided to the user when an activity level of the brain is increased;
a threshold computing subsystem configured to compute a threshold value at a plurality of time series in the neurofeedback session based on at least one of: the measured activity rate and the one or more digital biomarkers captured during the baseline activity of the brain, using an activity model; and
a neurofeedback output subsystem configured to output the neurofeedback signal corresponding to the neurofeedback session to the user, based on the computed threshold value.

8. The computer-implemented system as claimed in claim 7, wherein in computing the threshold value, the threshold computing subsystem, using the activity model, is configured to:
compute mean and standard deviation for each window, wherein the window in which the one or more digital biomarkers are stored:
detect a presence of an artifact in a biomarker list for the window, wherein the mean value and an adjacent factor are applied when the artifact is detected in the biomarker list, wherein applying of the mean value and the adjacent factor is ignored when recalibration for the one or more digital biomarkers is performed, and wherein the adjacent factor is related to a list of weights computed for weighted average of the biomarker list:
add a real-time value of a current biomarker to the biomarker list, wherein a list of differences between biomarker values in the biomarker list and a current threshold value, is determined using the adjacent factor:
determine whether the current biomarker continuously exceeds a second threshold value of a statistical range for more than recalibration count, wherein the recalibration count is related to a timer before recalibration is performed:
perform the recalibration for the one or more digital biomarkers when the current biomarker continuously exceeds the second threshold value of the statistical range for more than the recalibration count; and
compute mean and standard deviation for a next window when the current biomarker is within the second threshold value of the statistical range.

9. The computer-implemented system as claimed in claim 8, wherein the recalibration for the one or more digital biomarkers is performed by:
assigning weightage to the biomarker values based on an order in the plurality of time series, wherein the most recent digital biomarker value is assigned with higher weightage:
computing a weighted average for the biomarker values to identify the window to be prioritized, wherein the window is identified for computing a new threshold value, and wherein the window comprises a weighted average that is farthest from an initial threshold value obtains the highest priority; and
applying the computed new threshold value for the window comprising the one or more digital biomarkers for measuring the activity rate.

10. The computer-implemented system as claimed in claim 9, wherein the initial threshold value is computed from a list of the one or more digital biomarkers in the baseline activity.

11. The computer-implemented system as claimed in claim 10, wherein the initial threshold value at an initial time is a difference of mean values of the one or more digital biomarkers and X times standard deviation of the values of the one or more digital biomarkers, wherein the X times refer to Z-value from normal distribution that is computed based on the measured activity rate.

12. The computer-implemented system as claimed in claim 11, wherein the Z-value from normal distribution is computed from a standard table for the measured activity rate, and wherein the standard table is at least one of: a look-up table, and a hash table.

13. A non-transitory computer-readable storage medium having instructions stored therein that when executed by one or more hardware processors, cause the one or more hardware processors to execute operations of:
capturing one or more bio-signals representing as one or more digital biomarkers, wherein the one or more digital biomarkers indicates one or more brain activities of a user, and wherein the one or more brain activities of the user comprises rhythms and function of a brain:
measuring a baseline activity associated with the captured one or more digital biomarkers during a resting state of the brain;
measuring an activity rate based on the one or more digital biomarkers, wherein the activity rate comprises a positive feedback provided to the user when an activity level of the brain is increased:
computing a threshold value at a plurality of time series in the neurofeedback session based on at least one of: the measured activity rate and the one or more digital biomarkers captured during the baseline activity of the brain, using an activity model; and
outputting the neurofeedback signal corresponding to the neurofeedback session to the user, based on the computed threshold value.

14. The non-transitory computer-readable storage medium of claim 13, further comprising instructions to cause the processor to perform computation of the threshold value using the activity model by:
computing mean and standard deviation for each window, wherein the window in which the one or more digital biomarkers are stored:
detecting a presence of an artifact in a biomarker list for the window, wherein the mean value and an adjacent factor are applied when the artifact is detected in the biomarker list, and wherein applying of the mean value and the adjacent factor is ignored when recalibration for the one or more digital biomarkers is performed, wherein the adjacent factor is related to a list of weights computed for weighted average of the biomarker list:
adding a real-time value of a current biomarker to the biomarker list, wherein a list of differences between biomarker values in the biomarker list and a current threshold value is determined using the adjacent factor:
determining whether the current biomarker is continuously outside the statistical range for more than recalibration count, wherein the recalibration count is related to a timer before recalibration is performed:
performing recalibration for one or more digital biomarkers when the current biomarker is continuously outside the statistical range for more than the recalibration count; and
computing mean and standard deviation for a next window when the current biomarker is within the statistical range for more than the recalibration count.

15. The non-transitory computer-readable storage medium of claim 14, further comprising instructions to cause the processor to perform the recalibration for the one or more digital biomarkers by:
assigning weightage to the biomarker values based on an order in the plurality of time series, wherein the most recent digital biomarker value is assigned with higher weightage:
computing a weighted average for the biomarker values to identify the window to be prioritized, wherein the window is identified for computing a new threshold value, and wherein the window comprises a weighted average that is farthest from an initial threshold value obtains the highest priority; and
applying the computed new threshold value for the window comprising the one or more digital biomarkers for measuring the activity rate.

16. The non-transitory computer-readable storage medium of claim 15, wherein the initial threshold value is computed from a list of the one or more digital biomarkers in the baseline activity.

17. The non-transitory computer-readable storage medium of claim 16, wherein the initial threshold value at an initial time is a difference of mean values of the one or more digital biomarkers and X times standard deviation of the values of the one or more digital biomarkers, wherein the X times refer to Z-value from normal distribution that is computed based on the measured activity rate.

18. The non-transitory computer-readable storage medium of claim 17, wherein the Z-value from normal distribution is computed from a standard table for the measured activity rate, and wherein the standard table is at least one of: a look-up table, and a hash table.
